(19) United States
(12) Patent Application Publication
(10) Pub. No.: US 2005/0272808 A1
(43) Pub. Date: Dec. 8, 2005

(54) Compositions and methods for the treatment of hypertension

(76) Inventors: Nadav Zamir, Holon (IL); Boaz Musafia, Hod Hasharon (IL)

Correspondence Address: NATH & ASSOCIATES, PLLC, Sixth Floor, 1030 15th Street, N.W., Washington, DC, 20005, US

(21) Appl. No.: 11/126,407

(22) Filed: May 11, 2005

Related U.S. Application Data
(60) Provisional application No. 60/569,627, filed on May 11, 2004.

(51) Int. Cl. A61K 31/335

(52) U.S. Cl. 514/453; 514/462

(57) ABSTRACT

The present invention relates to use of 1,7-dioxaspiro[5.5]undecane and derivatives thereof for the preparation of a medicament for the treatment or prevention of hypertension. The invention also provides a method for treatment or prevention of hypertension using a medicament comprising 1,7-dioxaspiro[5.5]undecane or a derivative thereof as an active ingredient, such that the hypertension is treated or prevented, as the case may be.

### FIELD OF THE INVENTION

This invention relates to the treatment of hypertension. More specifically, the invention relates to a medicament for the treatment of hypertension.

### LIST OF REFERENCES

The following references are considered to be pertinent for the purpose of understanding the background of the present invention:
1. WO 01/39766
2. Williams G H, 2001 Approach to the Patient with Hypertension in Harrison's Principles of Internal Medicine, (15^{th} Edition. Publisher McGraw-Hill Companies Inc., Editor: Braunwald E) pp 211-214
3. Lucas K A, et al. 2000 Guanylyl Cyclases and Signalling by cyclyc GMP Pharmacol. Rev. 52:375-414
4. Ding et al. 1999. Activation ofparticulate guanylyl cyclase by endothelins in cultured SV-40 transformed cat iris sphincter smooth muscle cells. Life Sci. Vol: 64 pp. 161-174

### BACKGROUND OF THE INVENTION

Hypertension is the most common cardiovascular disease. It is defined as blood pressure higher than 140 mmHg systolic or 90 mmHg diastolic. Hypertension precedes development of congestive heart failure (CHF) in 91% of the cases and is a major risk factor for myocardial infarction and stroke [Williams, 2001].

Known antihypertensive drugs are classified according to their mechanism of action as diuretics, angiotensin-converting enzyme (ACE) inhibitors, alpha-adrenergic blockers, beta-adrenergic blockers, angiotensin II antagonists, calcium channel blockers and vasodilators. Each class of drugs has known side effects that sometimes prevents administration of the drugs to a given patient or class of patients. Moreover, of those with high blood pressure, 26.2% are on medication but their hypertension is not adequately controlled and 14.8% do not receive any medication for various reasons, mainly non compliance due to the drugs' side effects [Williams, 2001]. Thus, there is still and unmet need for new drugs with improved efficacy and reduced side effects.

Many of the cardiac, vascular, and renal responses in hypertension are mediated by signal transduction involving the second messenger cGMP (Guanosine 3′, 5′-cyclic monophosphate) [Lucas, 2000]. Endogenous and exogenous compounds, e.g., natriuretic peptides, nitric oxide (NO), and nitrates, modulate vasodilatation and blood pressure by increasing the intracellular concentration of cGMP in vascular smooth muscle cells, causing activation of the cGMP-dependent protein kinase (PKG) (Lucas, 2000). There are several mechanisms that result in elevation of intracellular cGMP levels such as activation of soluble guanylyl cyclase, activation of particulate guanylyl cyclase and inhibition of cGMP—dependent phosphodiesterase ((PDE) type I and V).

Semiochemicals-derived compounds such as Z-7-Tetradecenal, Z-13-Octadecenal, E,E-8,10-Dodecadienol, Z-11-Hexadecenyl-Acetate and 1,7-Dioxaspiro[5.5]undecane were shown to elevate cGMP levels in vitro in cultured SV40 transformed cat iris sphincter smooth muscle (SV-CISM-2) (WO 01/39766).

One of the problems in selecting an active ingredient for the reduction of blood pressure is that this reduction must be stringently controlled, since excessive lowering of blood pressure is also hazardous and may lead to death of the subject. Careful screening of potential active ingredients must be carried out to select only those that would lower systemic blood pressure with only acceptable side effects.

### SUMMARY OF THE INVENTION

The present invention is based on the discovery that 1,7-dioxaspiro[5.5]undecane lowered systemic blood pressure without causing discernible harm to the tested animals and without causing a detectable effect on heart contractility.

Thus, the present invention concerns the use of a compound of the general formula I: 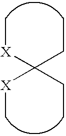
wherein
X represents, independently, an oxygen or a sulfur atom;
each ring represents, independently, a saturated or unsaturated, five, six or seven-membered ring; each ring may, independently, be substituted with one or more groups of the formula—RY or may be substituted with an exo-cyclic double bond in which one of the doubly bonded atoms is a carbon of the ring system and the other atom is selected from C, O or S;
wherein
R represents a valence bond or a linear or branched lower alkylene, lower alkenylene or lower alkynylene; and
Y represents a hydrogen, a halogen, —C(O)NZZ′, —C(O)—OZ or —OZ, wherein Z and Z′ may be the same or different and may represent each independently a hydrogen, a linear or branched alkyl, alkenyl or alkynyl; or
when both or one of the rings in said compound of the general formula I is substituted with at least two of the above substituents, said substitution being at neighboring carbon atoms, the two substituents, together with the carbon atoms to which they are bonded, may form a saturated or unsaturated five, six or seven-membered ring which may be carbocyclic or heterocyclic and which may be further substituted with the group —RY, as defined above.

In one embodiment, Z and Z′ may each independently represent a hydrogen, a linear or branched lower alkyl, lower alkenyl or lower alkynyl.

In another embodiment, in the compounds of general formula (I), X is oxygen and each of the two rings is a 6-membered ring. In such a system, the two oxygen atoms are at a 1,7 configuration.

In yet another embodiment of the present invention, the compounds are those having molecular weights smaller than about 500 grams per mole.

In still another embodiment, the compounds are those having molecular weights smaller than about 380 grams per mole.

The compounds of general Formula (I) which may be used in the accordance with the present invention are:
1,7-dioxaspiro[5.5]undecane;
1,7-dioxaspiro[5.5]undec-2-ene;
1,7-dioxaspiro[5.5]undec-3-ene;
1,7-dioxaspiro[5.5]undec-4-ene;
1,7-Dioxa-spiro[5.5]undecan-5-ol; and
1,7-Dioxa-spiro[5.5]undecan-4-ol.

The term “alkyl” as used herein refers to a straight or branched aliphatic hydrocarbon radical having up to 6 carbon atoms and includes for example methyl, ethyl, n-propyl, iso-propyl, n-butyl, iso-butyl, tert-butyl, n-heptyl, 2,2-dimethylpropyl, n-hexyl and the like. The term “alkylene” refers to such straight or branched aliphatic mid-chain radicals such as methylene, ethylene, propylene, butylenes and others.

The term “alkenyl” refers to straight or branched hydrocarbon radicals having between 2 and 6 carbon atoms and at least one double bond, preferably a terminal double bond, and includes for example vinyl, prop-2-en-1-yl, but-3-en-1-yl, pent-4-en-1-yl, and hex-5-en-1-yl. The term “alkenylene” refers to mid-chain radicals having at least one carbon-carbon double bond.

The term “alkynyl” refers typically to a hydrocarbon radical chain having between 2 and 6 carbon atoms and at least one triple bond. The term “lower” is used herein to denote alkyl, alkylene, alkenyl, alkenylene, alkynyl or alkynylene groups being C₁-C₄ or C₂-C₄ in length.

The term “halogen” refers to fluoro, chloro, bromo or iodo.

The group —C(O)NZZ′ may be —C(O)NH₂ when both Z and Z′ are hydrogens, or may be —C(O)NHZ′ when Z is a hydrogen and Z′ is an alkyl, alkenyl or alkynyl as defined above, or may be —C(O)N(ring) when both Z and Z′ together with the nitrogen atom to which they are attached, form a 5-7 memebred heterocyclic ring. The term “heterocyclic” or any lingual variation thereof, refers to a radical derived from a mono or polycyclic ring system having at least one atom selected from N, O and/or S, or to a radical derived from a mono or polycyclic heteroaromatic ring containing at least one atom selected from N, O and/or S. Such rings may for example be pyrrolidino, piperidino, morpholino, thiomorpholino, piperazino, alkylpiperazino, and the like.

The term “saturated” as used for example in reference to a ring system refers to a ring system which does not contain any double or triple bonds. However, while not falling literally within the above definition, compounds comprising exo-cyclic double bonds in which one of the doubly bonded atoms is a carbon of the ring system are also contemplated. The term “unsaturated” as used for example in reference to a ring system refers to a ring system which contains at least one double or triple bond, or to a ring system which is aromatic.

The term “exo-cyclic double bond” refers to a double bond which only one of the atoms thereof is part of the cycle. The second atom making the double bond may be a carbon atom (methylene group), oxygen atom (a keto group), or a sulfur atom (thioketo group).

The compound of the general formula I, according to the invention, may all be chiral in nature. Thus, within the scope of the present invention are contemplated also R and S (or ‘+’ and ‘−’) enantiomeric forms of said compounds, as well as mixtures thereof. The mixture may be a racemic mixture, meaning being comprised of both enantiomers at an equal ratio (50:50), or may contain one of the two enantiomers at an enantiomeric excess. One example of an enantiomer pair is the (R)-1,7-dioxaspiro[5.5]undecane form and the (S)-1,7-dioxaspiro[5.5]undecane form. Substitution at the dioxaspiro skeleton may afford further chiral centers, based on the substitution, which may further afford diastereomeric mixtures. These are also contemplated as suitable for use in the invention of the present application.

A person skilled in the art of the invention would appreciate that the scope of the present invention comprises any compound of the general formula (I) as defined above which would essentially have the same properties and effects as 1,7-dioxaspiro[5. 5]undecane.

Thus, according to a first aspect of the present invention use of a compound of the general formula (I) as defined above is provided for the preparation of a medicament for the treatment or prevention of hypertension.

The term “treatment” or any lingual variation thereof refers within the scope of the present invention to an administration of a medicament with the result that high blood pressure is reduced or cured. The reduction or curing as a result of treatment includes an effect on at least one parameter of hypertension, including systolic and/or diastolic blood pressure or any of the resulting effects known to be associated with hypertension.

The term “prevention” or any lingual variation thereof refers within the scope of the present invention to an administration of a medicament with the result that the subject's blood pressure (systolic and/or diastolic) is maintained within a desired range. Generally, blood pressure is considered normal or desired if the systolic blood is 130 mmHg or less and the diastolic blood pressure is 85 mHg or less Categories of blood pressure are detailed in Table 1 [Williams, 2001].

TABLE 1
Classification of blood pressure for adults aged 18 years and older

| | Systolic pressure | Diastolic pressure |
|---|---|---|
| Category | (mmHg) | (mmHg) |
| Optimal | <120 | <80 |
| Normal | <130 | <85 |
| High Normal | 130-139 | 85-89 |
| Hypertension | | |
| Mild | 140-159 | 90-99 |
| Moderate | 160-179 | 100-109 |
| Severe | 180-209 | 110-119 |

However, for many patients other parameters may affect the desired blood pressure and thus it may be desirable to maintain it at above-normal or below-normal pressures. For example in some cases, such as some geriatric patients, the desirable blood pressure is that of mild hypertension (systolic 140-159 mmHg and diastolic 90-99 mmHg).

According to another aspect, the present invention provides a method for the treatment of hypertension comprising administering to an individual an effective amount of a medicament comprising a compound of the general formula I as defined above as an active ingredient, such that the hypertension is treated.

According to yet another aspect, the present invention provides a method for the prevention of hypertension comprising administration to an individual an effective amount of a medicament comprising a compound of the general formula I as defined above as an active ingredient, such that the blood pressure is maintained substantially within a desirable rate.

In the context of this invention an individual's blood pressure is within a desirable range if the systolic and/or diastolic blood pressure is within the desired category as described in table 1 (e.g. normal blood pressure, high normal blood pressure, mild hypertension, etc).

According to yet another aspect, the present invention provides a medicament for the treatment or prevention of hypertension comprising a compound of the general formula I as defined above as an effective ingredient and a pharmaceutically acceptable carrier.

The “effective amount” for purposes herein is determined by such considerations as may be known in the art. The amount must be effective to achieve the desired therapeutic effect (i.e. treatment or prevention) as described above. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half life in the body, on undesired side effects, if any, on factors such as age, gender, family history, weight etc.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a graphic representation of the effect of two bolus injections of 1,7-dioxaspiro[5.5]undecane on systemic blood pressure. Arrows indicate the times of bolus injections. FIG. 1A depicts the changes in systolic blood pressure, and FIG. 1B depicts the changes in diastolic blood pressure.

FIG. 2 is a graphic representation of the effect of two bolus injections of Z-13-octadecenal on systemic blood pressure. Arrows indicate the times of bolus injections. FIG. 2A depicts the changes in systolic blood pressure, and FIG. 2B depicts the changes in diastolic blood pressure.

FIG. 3 is a graphic representation of the effect of i.v. bolus injection of 1,7-dioxaspiro[5.5]undecane (20 mg/Kg) on systemic blood pressure in Spontaneously Hypertensive Rats (SHR). FIG. 3A depicts the changes in systolic blood pressure and FIG. 3B depicts changes in diastolic blood pressure.

FIG. 4 is a graphic representation of the effect of oral administration of 1,7-dioxaspiro[5.5]undecane on systemic blood pressure Spontaneously Hypertensive Rats (SHR). FIG. 4A depicts changes in systolic blood pressure and FIG. 4B depicts changes in diastolic blood pressure.

FIG. 5 is a graphic representation of the effect of i.v. bolus administration of 1,7-dioxaspiro[5.5]undec-2-ene on systemic blood pressure Spontaneously Hypertensive Rats (SHR). FIG. 5A depicts changes in systolic blood pressure and FIG. 5B depicts changes in diastolic blood pressure.

### DETAILED DESCRIPTION OF THE INVENTION

According to a first aspect of the present invention use of a compound of the general formula (I) as defined above is provided for the preparation of a medicament for the treatment of hypertension.

The medicament may be administered to an individual by one of a variety of administration modes, including, but not limited to oral, intravenous, intramuscular, transdermal, subcutaneous, topical, sublingual, rectal, nasal, and the like. Additionally, the medicament may be administered by inhalation.

When the medicament is administered intravenously, it may be in form of an infusion (e.g. continuously) or injection, such as bolus injection. When the medicament of the invention is administered orally, it may be administered in the form of a tablet, a pill, a capsule (e.g. a gelatin capsule) a powder or a pellet. Where a liquid carrier is used, the oral preparation may be in the form of a syrup, emulsion, or soft gelatin capsule. Nasal administration may be by nasal insufflation or as an aerosol, and internal administration such as rectal administration may be by use of a suppository. Compositions for topical administration may be, for example, in the form of creams, ointments, lotions, solutions, gels and transdermal patches.

The compound according to Formula (I) as defined above may be typically be administered with a pharmaceutically acceptable carrier which does not interfere with the efficacy of the active ingredient. The carrier may be selected from a large number of carriers known in the art and its nature will depend on the intended form of administration and indication for which the active ingredient is used.

Tablets, pills and capsules containing the compound according to Formula (I) as defined above may also include conventional excipients such as lactose, starch, magnesium stearate, and the like. Suppositories may include excipients such as waxes and glycerol. Injectable solutions may comprise saline, buffering agents, dextrose, water, glycerol, ethanol and solvents such as propylene glycol, polyethylene glycol and ethanol. Such solutions may also comprise stabilizing agents and preservatives which are typically antimicrobial agents (such as chlorbutol, benzyl alcohol, sodium benzoate, ascorbic acid, and phenol) and antioxidants (such as butylated hydroxy toluene, propyl gallate, and sulfites). Enteric coatings, flavorings, and dyes and colorants may also be used.

At times, the medicaments of the present invention may be incorporated within a liposome prepared by any of the methods known in the art. In addition, the medicaments containing the compound according to Formula (I) as defined above may be encapsulated in inert polymerized particles such as, for example, nano particles, microspheres, and microparticles known in the art.

The medicament produced according to the invention may comprise the compound according to Formula (I) as defined above as the only active ingredient. Alternatively other active ingredients may be added. Such addition may for example exert a synergistic effect or reduce the side effects of each active ingredient.

According to the present invention, the medicament comprising the compound according to Formula (I) as defined above may be administered separately or, alternatively, in combination with various other treatments administered to the patient for the same or different disease, condition or symptom thereof. In case of combination with other treatments administered to the patient, the method or medicament of the present invention may be administered at the same time with, before and/or after the other treatment or medicament.

The effective amount of the compound according to Formula (I) as defined above administered for the treatment of Hypertension will be typically in the range of from about 0.1 to 200 mg/kg/day, or even from about 1.0 to 180 mg/kg/day. Lower dosage can be used also to obtain a transient short term effect.

It would be appreciated that in the methods of the present invention, an individual that is to be subject of treatment should be identified. For treatment of hypertension, such subject would be manifesting hypertension (either occasionally or chronically). For the purpose of prevention of hypertension would be such that is considered likely to suffer from hypertension. A person skilled in the art of the invention would know what parameters may be indicative of such likelihood. For example this may be based on the individual's medical history (related to hypertension or any other medical condition or parameter) or that of the individual's family, the individual's occupation, etc. Prevention of hypertension in case of a hypertensive individual may also be the prevention of an increase of hypertension. In such case the desired rate may be hypertensive as well.

According to yet another aspect, a medicament for the treatment or prevention of hypertension comprising the compound according to Formula (I) as defined above as an effective ingredient and a pharmaceutically acceptable carrier. This medicament may be used in the method of this invention and may be prepared and have any form as described in this application or known in the art.

Also contemplated by the present invention are pharmaceutically acceptable salts of the compounds of Formula (I), which may be formed by known methods with metals or amines, such as alkali and alkaline earth metals or organic amines. The salts may be prepared based on the existence of a reactive group at the main ring system or on a substituents attached thereto.

Generally speaking, acid addition salts of the compounds of general Formula (I) include salts derived from inorganic acids such as hydrochloric, nitric, phosphoric, sulfuric, hydrobromic, hydroiodic, and the like, as well as salts derived from organic acids such as aliphatic mono- or dicarboxylic acids, phenyl substituted alkanoic acids, hydroxyl alkanoic acids, aromatic acids, sulfonic acids, etc. Such salts may for example include sulfate, pyrosulfate, bisulfate, sulfite, bisulfite, nitrate, phosphate, metaphosphate, chloride, bromide, iodide, acetate, propionate, isobutyrate, oxalate, malonate, succinate, fumarate, maleate, benzoate and others. Also contemplated are salts of amino acids such as arginate and the like.

Formulations comprising one or more of the compounds disclosed herein may be prepared by admixing the compound of choice with appropriate excipients or diluents as was described hereinbefore and as known to a person versed in the art. Within the scope of the present invention, numerous formulations have been prepared. For example, a 100 ml stock formulation for i.v. administration may be prepared by admixing 5.0 g/100 ml of 1,7-dioxaspiro[5.5]undecane, 101.0 g/100 ml PEG-400 and 5.0 g/100 ml of Tween-20. Similarly, for a 200 ml stock formulation for p.o. administration may be prepared by admixing 20.0 g/100 ml 1,7-dioxaspiro[5.5]undecane, 53.3 g/100 ml MCT (medium chain triglyceride), 10.7 g/100 ml Tween-80 and 16.0 g/100 ml Tween-85.

Typically, prior to administration, the formulations were diluted in 5% glucose in a ratio of 1:4 for i.v. administration or in a ratio of 1:3 for p.o. administration.

### EXAMPLES

I. Materials

Anantin, L-NAME (N^{G}-nitro-L-arginine methyl ester) and IBMX (3-isobutyl-methylxanthine), were purchased from Sigma Chemicals Co. St. Louis, Mo., USA. [¹²⁵I] cGMP RIA kit was purchased from Amersham, Arlington Heights, Ill., USA. Reagents for cell cultures were purchased from GIBCO Life Technologies, Grand Island, N.Y., USA. All other chemicals were likewise fine laboratory grade chemicals purchased periodically from established manufacturers.

1,7-dioxaspiro[5.5]undecane and Z-13-octadecenal were dissolved in ethanol and diluted with saline prior to intravenous administration. The dioxaspiro compounds were also administered as part of pre-made formulations as was described hereinbefore.

II. In-Vivo Procedures

Compounds that were shown to be able to elevate cGMP levels in cultured smooth muscle cells (WO 01/39766) were tested for their ability to lower blood pressure of normotensive rats. These were 1,7-dioxaspiro[5.5]undecane and another compound termed Z-13-octadecenal. Both compounds showed comparable ability to elevate cGMP levels in smooth cultured muscle cells (WO 01/39766).

A. Male Spargue-Dawley Rats

1. Rat Strain and Experimental Preparations

Male Sprague-Dawley rats, weighing 250-350 grams were anaesthetized with mixture of Ketamine (90 mg/Kg body weight) and Xylazine (10 mg/Kg body weight) in a single bolus injection. The rats' chest was shaven; and the rats were secured in supine position. Anaesthetized rats were intubated with 18G catheter, connected to a ventilator [Harvard Apparatus] and ventilated with a room air (volume: 2 ml, rate: 70 per min). The left femoral vein was cannulated for drug administration, which is standardized to injections of 0.2 ml/100 grams body weight over a period of 0.5-1 minutes.

For measurement of hemodynamic parameters [blood pressure (BP), heart rate (HR)] a cannula was inserted retrogradely into the left carotid artery. The tip of the catheter was positioned close to the origin of the subclavian artery.

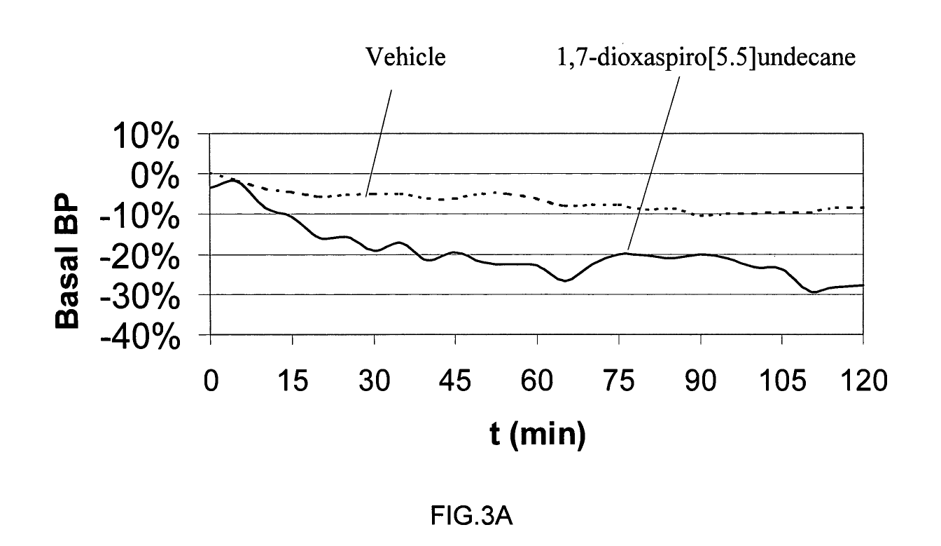

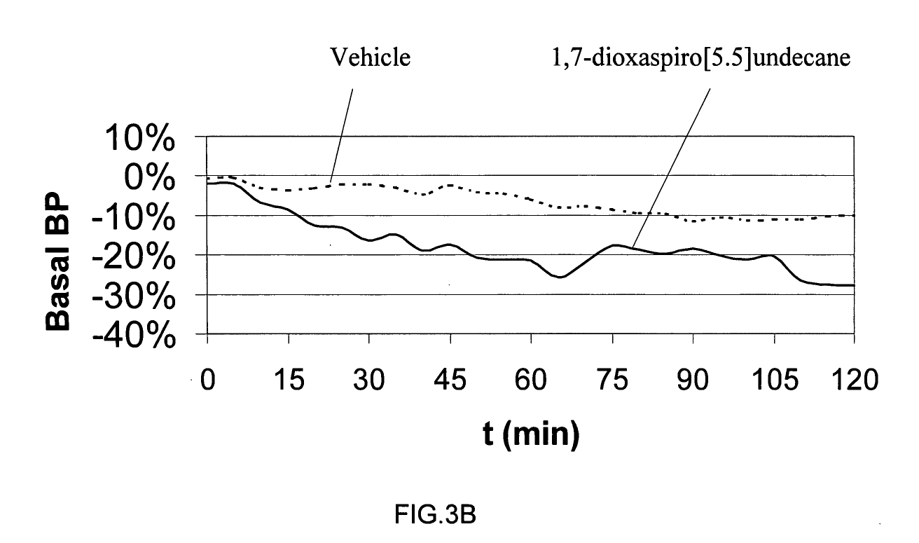

Similar experiments were conducted using the Spontaneously Hypertensive Rats (SHR) strain. The results of this study are presented in FIGS. 3 to 5.

2. Continuous Monitoring of ECG and Hemodynamic Parameters

For continuous monitoring of blood pressure (systolic and diastolic pressure) and heart rate, the catheter was connected to a pressure transducer [Grass instrument co., Quincy, Mass. USA]. To monitor the ECG (electrocardiogram): two electrodes were connected to the animal's arms and one electrode was connected to its left leg. The monitoring system was calibrated at the beginning of each experiment. Recording all the steps of the calibration. ECG and hemodynamic parameters were recorded continuously during the whole experiment.

Monitoring rate (speed of paper) was at 5 mm/sec. At different times (stabilization, after injections, blood pressure changes, etc.,) the monitoring rate was changed to 25 mm/sec. In addition routinely, every 5 minutes the monitoring rate was changed to 25 mm/sec. for 15 sec. ECG recordings were obtained at the same times as hemodynamic measurements.

3. Test Compound Administration

When stable hemodynamic conditions were achieved for at least 20 minutes (baseline control values), a test compound (1,7-dioxaspiro[5.5]undecane or Z-13-octadecenal) was administrated twice. The test compound was administered by bolus injection intravenously at different doses (2,4,6,8 mg/Kg body weight). Each dose was administrated twice in the same manner. The second dose was given after the hemodynamic parameters were recovered (or stabilized) following the first injection.

B. Spontaneously Hypertensive Rats

1. Rat Strain and Experimental Preparations and Continuous Monitoring of ECG and Hemodynamic Parameters

Spontaneously Hypertensive Rats, (Harlan USA) 290-330 gr weight, were anaesthetized with 100-120 mg/kg Inactin in one single bolus injection i.p. The chest was shaven, and animal was secured in supine position. Anaesthetized rats were subject to tracheotomy with 18G catheter. The left femoral vein was cannulated for drug administration and infusion.

For measurement of hemodynamic parameters a cannula was inserted retrogradely into the left carotid artery. The tip of the catheter was positioned close to the origin of the subclavian artery. The catheter was connected to a blood pressure measurement system (BioPac MP100 workstation, BioPac System inc.). ECG was recorded by BioPac MP100 workstation. The information was saved electronically.

During the experiment 1 ml/h of Hartmann's solution was infused i.v.

2. Test Compound Administration

When stable hemodynamic conditions were achieved for about 20 minutes (baseline control values), test or control treatment was administrated bolus injection 20 mg/Kg i.v. of the PEG-based formulation. Alternatively an 80-125 mg/Kg of MCT-based formulation was injected p.o. intra stomach.

C. Results

Effect of 1,7-dioxaspiro[5.5]undecane on Systemic Blood Pressure and ECG of Normotensive Anesthetized Rats

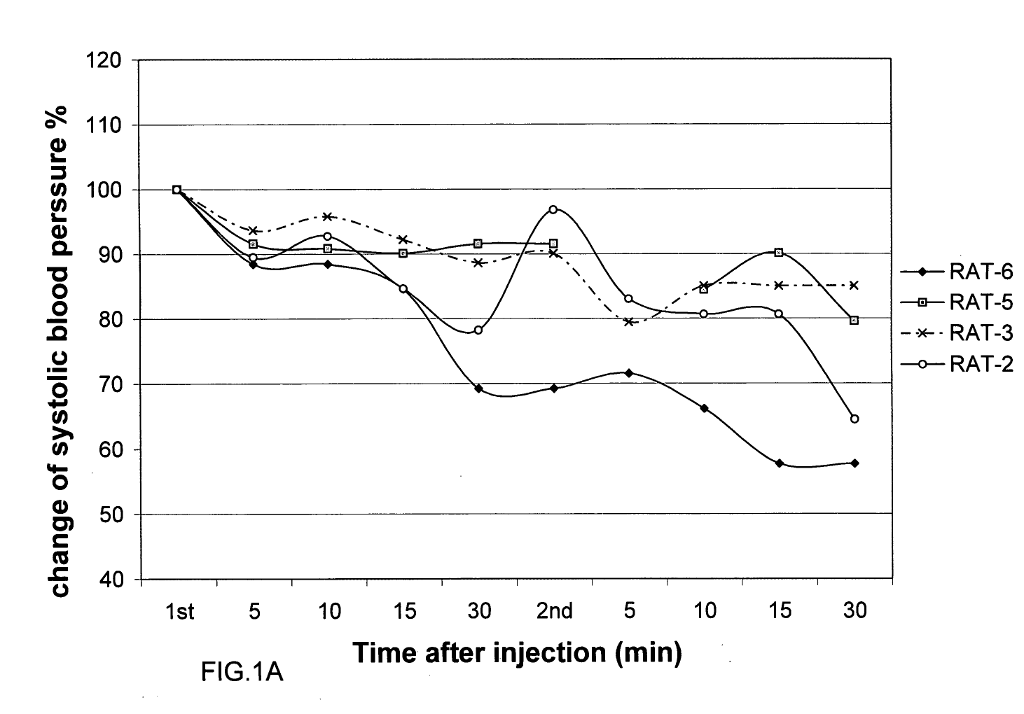

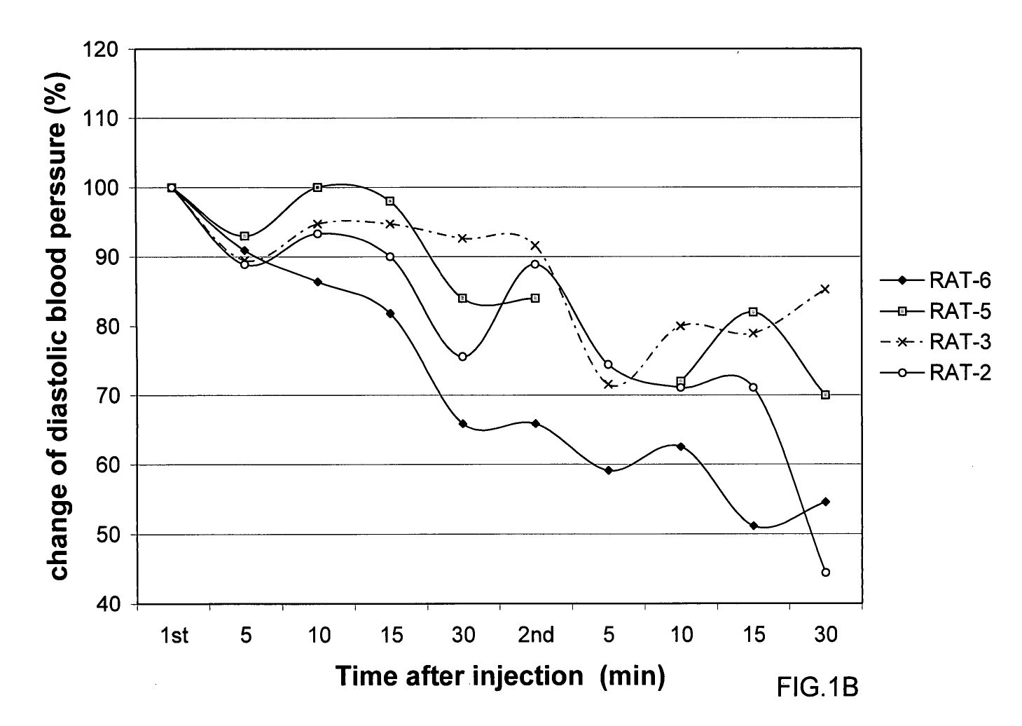

The effect of 1,7-dioxaspiro[5.5]undecane (at different doses, and multiple dosing) on systemic blood pressure is shown in FIG. 1 as well as in FIGS. 3 to 5.

As can be seen in FIG. 1A bolus injection of 1,7-dioxaspiro[5.5]undecane (8 mg/Kg body weight) caused after 30 minutes a reduction in systolic blood pressure that ranged between 10-30% (average 18.1%). Additional bolus injection of 1,7-dioxaspiro[5.5]undecane at the same dose caused after 30 minutes a greater reduction in systolic blood pressure that ranged between 15-42% (average 28.3%).

Similarly, as shown in FIG. 1B, 1,7-dioxaspiro[5.5]undecane (8 mg/Kg body weight) caused after 30 minutes a reduction in diastolic blood pressure that ranged between 7-32% (average 20.5%). Additional bolus injection of 1,7-dioxaspiro[5.5]undecane at the same dose caused after 30 minutes a greater reduction in diastolic blood pressure that ranged between 15-53% (average 36.5%).

The effect of 1,7-dioxaspiro[5.5]undecane on systemic blood pressure at lower doses was measured as well.

Single and double dose of 1,7-dioxaspiro[5.5]undecane at 2 mg/Kg body weight had no effect on systemic blood pressure of anesthetized normotensive rats.

Single dose of 1,7-dioxaspiro[5.5]undecane at 4 mg/Kg body weight had a mild hypotensive effect that last for about 15 minutes on systemic blood pressure of anesthetized normotensive rats. This hypotensive effect was more pronounced after the second injection of the same dose.

1,7-dioxaspiro[5.5]undecane after single and double dosage of 2-8 mg/Kg body weight i.v. (intravenous) bolus injections had no effect on heart contractility was evidenced by ECG monitoring during the experiments, (data not shown). Thus it seems that 1,7-dioxaspiro[5.5]undecane exerts its vasodilatory effect via reduction in total peripheral resistance (TPR).

Effect of Z-13-octadecenal on Systemic Blood Pressure of Normotensive Rats

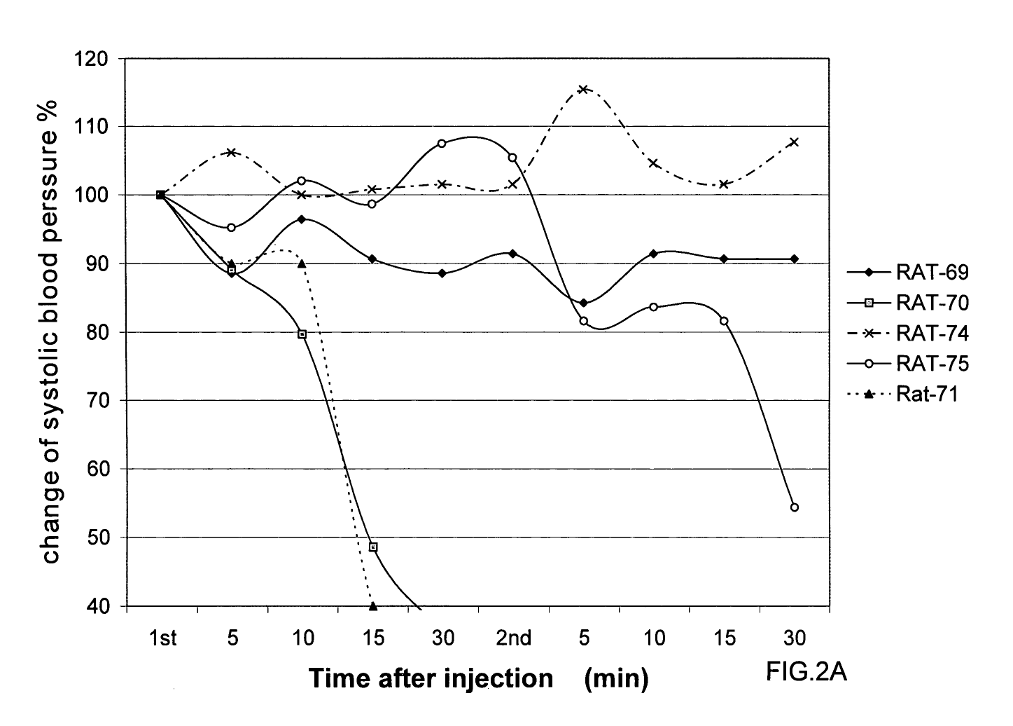

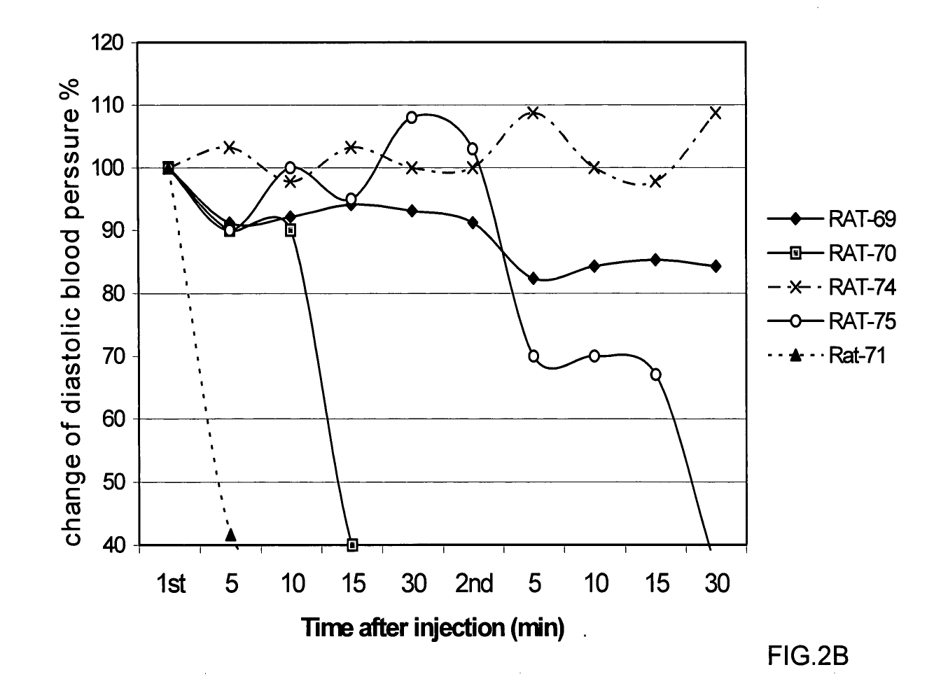

As can be seen in FIG. 2, bolus injection of 8-mg/Kg body weight Z-13-octadecenal caused an uncontrolled reduction in blood pressure in three out of five animals tested. The hypotensive/toxic effect was rapid and profound and lead to death of two animals within 15 minutes after the first injection of Z-13-octadecenal. After the second injection of Z-13-octadecenal a third animal exhibited a profound drop in systemic blood pressure that lead to death within 15 minutes. Interestingly, the two animals that survived showed minimal or no response of systemic blood pressure to both injections of Z-13-octadecenal.

Bolus injection of 4-mg/Kg body weight Z-13-octadecenal caused no reduction in blood pressure in two animal tested, in single and double dose administration. (data not shown)

Thus it is concluded that Z-13-octadecenal is a potent toxic compound that its intravenous administration resulted in death of three out of five animals tested.

Effect of 1,7-dioxaspiro[5.5]undecane on Systemic Blood Pressure of Anesthetized Spontaneously Hypertensive Rats

The effect of 1,7-dioxaspiro[5.5]undecane at 20 mg/Kg on systemic blood pressure of SHRs (9 placebo and 8 test subjects) is shown in FIG. 3. As may be observed, bolus injection of 1,7-dioxaspiro [5.5]undecane (20 mg/Kg body weight) caused 30 minutes after injection a reduction in systolic blood pressure that ranged between 20-30%. FIG. 3B shows similar affect on the diastolic blood pressure wherein a reduction of between 15-28% was observed. The maximal effect was 110-120 minutes after the injection. The effect lasted for least 90 minutes.

Effect of 1,7-dioxaspiro[5.5]undec-2-ene on Systemic Blood Pressure of Anesthetized Spontaneously Hypertensive Rat

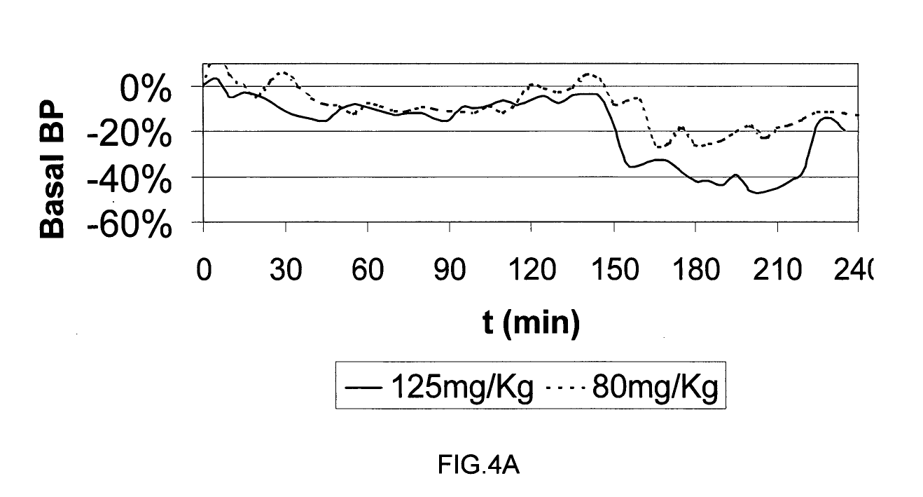

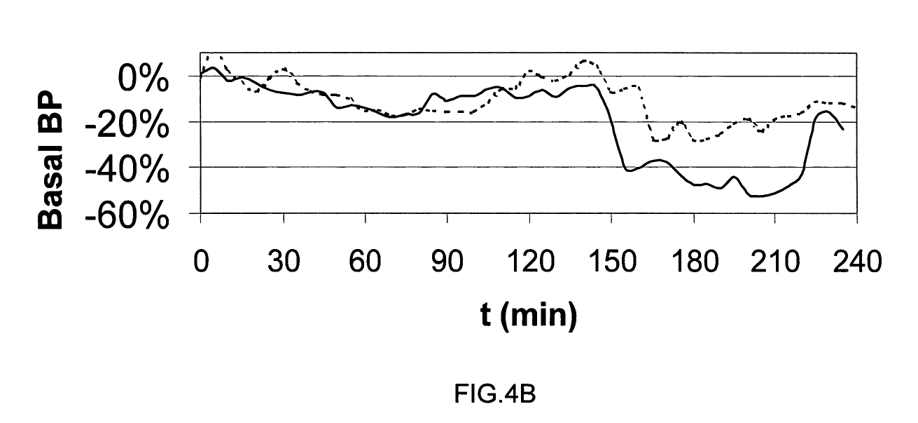

The effect of 1,7-dioxaspiro[5.5]undec-2-ene at 20 mg/Kg on systemic blood pressure of SHRs is shown in FIG. 4. As may be observed, bolus injection of 1,7-dioxaspiro[5.5]undec-2-ene (20 mg/Kg body weight) caused 30 minutes after injection a reduction in systolic blood pressure that ranged between 18-28%. FIG. 3B shows a similar affect on the diastolic blood pressure wherein a reduction of between 20-33% was observed. The maximal effect was at 35-55 minutes after the injection. The effect lasted about 30 minutes.

Effect of 1,7-dioxaspiro[5.5]undecane on Systemic Blood Pressure of Anesthetized Spontaneously Hypertensive Rats, Oral Administration

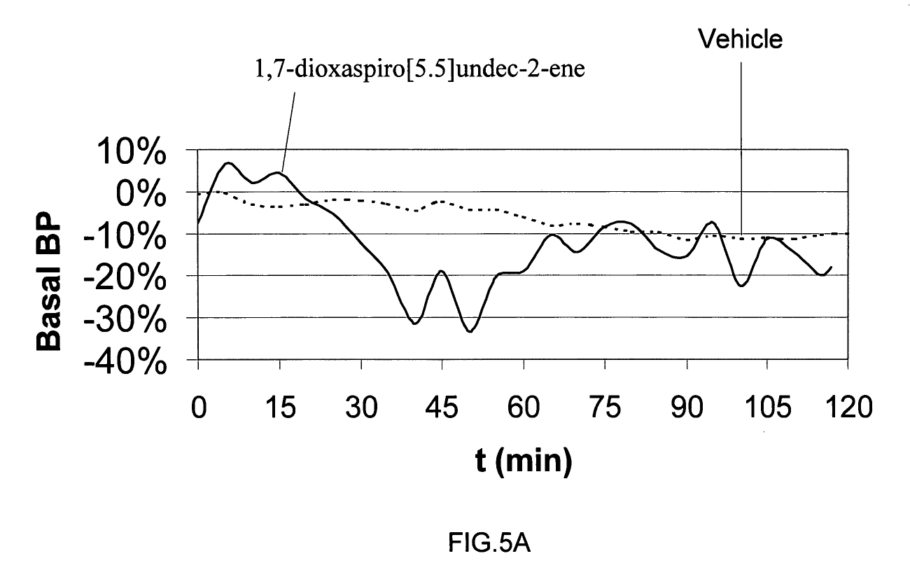

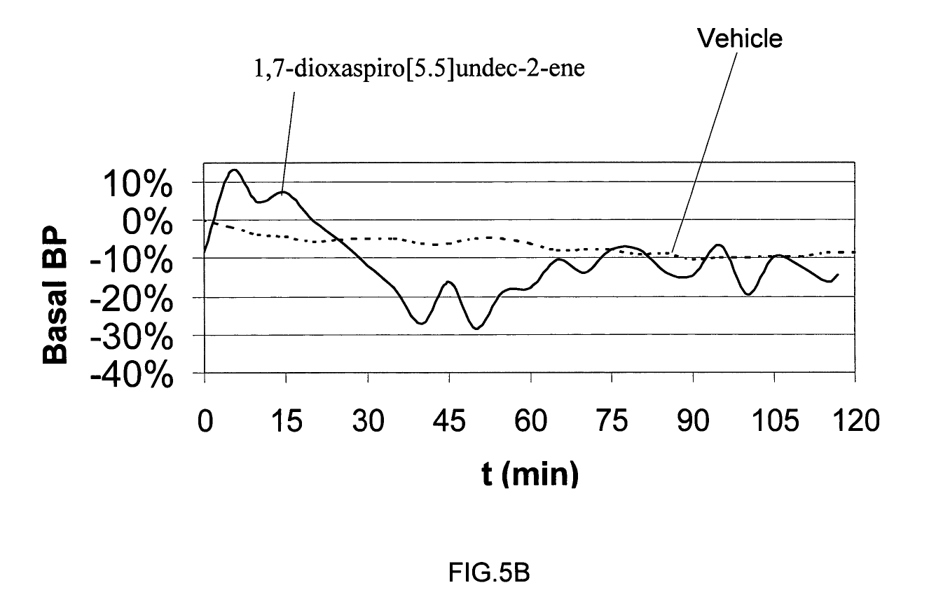

The effect of 1,7-dioxaspiro[5.5]undecane at 80 mg/Kg (doted line) or 125 mg/Kg (full line) on systemic blood pressure of SHRs is shown in FIG. 5. As can be seen, bolus injection of 125 mg/Kg of 1,7-dioxaspiro[5.5]undecane caused 150 minutes post injection a reduction in systolic blood pressure that ranged between 30-45%. Treatment with 80 mg/Kg body weight caused after 150 minutes a reduction in systolic blood pressure that ranged between 15-28%. FIG. 5B shows enhanced affect on the diastolic blood pressure. Bolus injection of 1,7-dioxaspiro[5.5]undecane 125 mg/Kg caused after 150 minutes a reduction in systolic blood pressure that ranged between 40-53%. Treatment with 80 mg/Kg caused after 150 minutes a reduction in systolic blood pressure that ranged between 20-30%. The maximal effect was at 150-220 minutes after the injection. The effect lasted about an hour.

III. In-Vitro Methods

The mechanism by which 1,7-dioxaspiro[5.5]undecane elevates intracellular cGMP was elucidated as follows:

1. Cell Culture

Cat iris sphincter smooth muscle (SV-CISM-2) cells were maintained in Dulbecco's modified Eagle medium (DMEM), supplemented with 10% fetal calf serum (FCS) and 50 μg/ml gentamicin (culture medium) as described by Ding et al. 1999. (Life Sci. Vol: 64 pp. 161-174)

To initiate subculture, confluent SV-CISM-2 cells were washed with Ca²⁺-Mg²⁺ free Dulbecco's phosphate buffered salt solution (PBS) and treated with 0.05% trypsin in 0.5 mM EDTA for 3 minutes at 37° C. This was followed by addition of culture medium, and the cell suspension was centrifuged at 200 g for 5 minutes. The pelleted cells were suspended in culture medium and seeded in 6-well plates for the cGMP assay.

2. cGMP Assay

Confluent cells, in 6-well plates were washed once with DMEM and incubated in 2 ml DMEM for 15 minutes at 37° C. IBMX (0.1 mM) was added and incubated for additional 10 minutes. The tested agent was then added and incubations continued for 10 minutes. Anantin (1 μM) or L-NAME (0.1 mM) were added along with IBMX, 10 minutes prior to addition of the tested compound. Reactions were stopped by replacing the medium with 1 ml ice-cold 5% trichloroacetic acid (TCA) (W/V).

The obtained samples were homogenized, centrifuged and the supernatants were extracted with anhydrous diethylether. A 100 μl portion of the supernatant from each sample was taken; and after appropriate dilutions cGMP was assayed by radioimmunoassay RIA as described by the aforementioned Ding et al. 1999.

3. Determination of Protein

Protein content was determinate by the method of Lowry with bovine serum albumin as standard as described in the aforementioned Ding et al. 1999.

4. Data

The data are presented as mean±SEM of three separate experiments with triplicate incubations for each data point.

5. Results

L-Arginine analogue (L-NAME), an NO synthase inhibitor, was used to test the involvement of soluble guanylyl cyclase. Anantin, a specific membranal guanylyl cyclase antagonist, was used to examine the role of this enzyme in 1,7-dioxaspiro[5.5]undecane action. The experiments with 1,7-dioxaspiro[5.5]undecane were preformed in presence of IBMX thus excluding PDE inhibition by 1,7-dioxaspiro[5.5]undecane as a mechanism for cGMP elevation.

The levels of cGMP were measured, and the results of different treatments are depicted in Table 1.

TABLE 2
The mechanism of 1,7-dioxaspiro[5.5]undecane
stimulating cGMP production in-vitro

| | cGMP formed | |
|---|---|---|
| | (pmol/mg protein/ | cGMP formed |
| Treatment | 10 min) | % of control |
| Control | 0.52 ± 0.07 | 100 |
| 1,7-dioxaspiro[5.5]undecane | 1.23 ± 0.1 | 236 |
| (0.1 μM) | | |
| 1,7-dioxaspiro[5.5]undecane | 1.32 ± 0.1 | 253 |
| (0.1 μM) & Anantin (1 μM) | | |
| 1,7-dioxaspiro[5.5]undecane | 0.94 ± 0.15 | 182 |
| (0.1 μM) & L-NAME (100 μM) | | |

As seen in Table 1,1,7-dioxaspiro[5.5]undecane increased cGMP concentration by 2.36 times.

1,7-dioxaspiro[5.5]undecane did not elevate cGMP via activation of membranal guanylyl cyclase since anantin (a specific membranal guanylyl cyclase antagonist) had no inhibitory effect on cGMP formation when tested together with 1,7-dioxaspiro[5.5]undecane. On the other hand the NO-synthase inhibitor, L-NAME, attenuated 1,7-dioxaspiro[5.5]undecane stimulatory effect on cGMP production. The chemical nature of 1,7-dioxaspiro[5.5]undecane indicates that this molecule cannot be a NO donor, there is no nitrogen atom in the molecular structure.

Based on the above information it is understood that 1,7-dioxaspiro[5.5]undecane may exert its action at least in part, via activation of soluble guanylyl cyclase.

1. A method for the reduction of hypertension comprising administration to an individual an effective amount of a medicament comprising a compound of the general formula I: 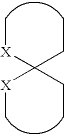
wherein
X represents, independently, oxygen or a sulfur atom;
each ring represents, independently, a saturated or unsaturated five, six or seven-membered ring; each ring may, independently, be substituted with one or more groups of the formula —RY or may be substituted with an exo-cyclic double bond in which one of the doubly bonded atoms is a carbon of the ring system and the other atom selected from C, O or S,
wherein
R represents a valence bond or a linear or branched lower alkylene, lower alkenylene or lower alkynylene; and
Y represents a hydrogen, a halogen, —C(O)NZZ′, —C(O)—OZ or —OZ, wherein Z and Z′ may be the same or different and may represent each independently a hydrogen or a linear or branched alkyl, alkenyl or alkynyl; or
when both or each ring in said compound of the general formula I is substituted with at least two of the above substituents, said substitution being at neighboring carbon atoms, the two substituents, together with the carbon atoms to which they are bonded, may form a saturated or unsaturated five, six or seven-membered ring which may be carbocyclic or heterocyclic and which may be substituted with the group —RY; as an active ingredient, such that the hypertension is treated.

2. The method of claim 1, wherein the amount of the active ingredient is in the range of from about 0.1 to 200 mg/kg/day.

3. The method of claim 2, wherein the amount of the active ingredient is in the range of from about 1.0 to 180 mg/kg/day.

4. The method of claim 3, wherein the medicament is injected intravenously or given orally.

5. The method of claim 1, wherein the medicament exerts its action, at least in part via activation of soluble guanylyl cyclase.

6. The method of claim 1, wherein X is oxygen and each of said rings is a 6-membered ring.

7. The method of claim 6, wherein the active ingredient is selected from 1,7-dioxaspiro[5.5]undecane, 1,7-dioxaspiro[5.5]undec-2-ene, 1,7-dioxaspiro[5.5]undec-3-ene, 1,7-dioxaspiro[5.5]undec-4-ene, 1,7-dioxaspiro[5.5]undecan-4-ol and 1,7-dioxaspiro[5.5]undecan-5-ol.

8. A method for the prevention of hypertension comprising administration to an individual an effective amount of a medicament comprising a compound of the general formula I: 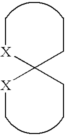
wherein
X represents, independently, oxygen or a sulfur atom;
each ring represents, independently, a saturated or unsaturated five, six or seven-membered ring; each ring may, independently, be substituted with one or more groups of the formula —RY or may be substituted with an exo-cyclic double bond in which one of the doubly bonded atoms is a carbon of the ring system and the other atom selected from C, O or S,
wherein
R represents a valence bond or a linear or branched lower alkylene, lower alkenylene or lower alkynylene; and
Y represents a hydrogen, a halogen, —C(O)NZZ′, —C(O)—OZ or —OZ, wherein Z and Z′ may be the same or different and may represent each independently a hydrogen or a linear or branched alkyl, alkenyl or alkynyl; or
when each ring in said compound of the general formula I is substituted with at least two of the above substituents, said substitution being at neighboring carbon atoms, the two substituents, together with the carbon atoms to which they are bonded, may form a saturated or unsaturated five, six or seven-membered ring which may be carbocyclic or heterocyclic and which may be substituted with the group —RY; as an active ingredient, such that the blood pressure is maintained substantially within a desirable rate.

9. The method of claim 8, wherein the amount of the active ingredient is in the range of from about 0.1 to 200 mg/kg/day.

10. The method of claim 9, wherein the amount of the active ingredient is in the range of from about 1.0 to 180 mg/kg/day.

11. The method of claim 10, wherein the medicament injected intravenously or given orally.

12. The method of claim 11, wherein the medicament exerts its action, at least in part via activation of soluble guanylyl cyclase.

13. The method of claim 8, wherein X is oxygen and each of said rings is a 6-membered ring.

14. The method of claim 13, wherein the active ingredient is selected from 1,7-dioxaspiro[5.5]undecane 1,7-dioxaspiro[5. 5]undec-2-ene, 1,7-dioxaspiro[5.5]undec-3-ene, 1,7-dioxaspiro[5.5]undec-4-ene, 1,7-dioxaspiro[5.5]undecan-4-ol and 1,7-dioxaspiro[5.5]undecan-5-ol.

15. A method for the reduction of hypertension comprising administration to an individual an effective amount of a medicament comprising 1,7-dioxasipro[5.5]undecane or 1,7-dioxasipro[5.5]undec-2-ene as an active ingredient, such that the hypertension is treated.

16. A method for the prevention of hypertension comprising administration to an individual an effective amount of a medicament comprising 1,7-dioxasipro[5.5]undecane or 1,7-dioxasipro[5.5]undec-2-ene as an active ingredient, such that the blood pressure is maintained substantially within a desirable rate.

17. A medicament for the treatment or prevention of hypertension comprising a compound of the general formula I: 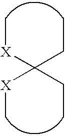
wherein
X represents, independently, oxygen or a sulfur atom;
each ring represents, independently, a saturated or unsaturated five, six or seven-membered ring; each ring may, independently, be substituted with one or more groups of the formula —RY or may be substituted with an exo-cyclic double bond in which one of the doubly bonded atoms is a carbon of the ring system and the other atom selected from C, O or S,
wherein
R represents a valence bond or a linear or branched lower alkylene, lower alkenylene or lower alkynylene; and
Y represents a hydrogen, a halogen, —C(O)NZZ′, —C(O)—OZ or —OZ, wherein Z and Z′ may be the same or different and may represent each independently a hydrogen or a linear or branched alkyl, alkenyl or alkynyl; or
when each ring in said compound of the general formula I is substituted with at least two of the above substituents, said substitution being at neighboring carbon atoms, the two substituents, together with the carbon atoms to which they are bonded, may form a saturated or unsaturated five, six or seven-membered ring which may be carbocyclic or heterocyclic and which may be substituted with the group —RY; as an effective ingredient and a pharmaceutically acceptable carrier.

18. The medicament of claim 17, wherein the amount of the active ingredient is in the range of from about 0.1 to 200 mg/kg/day.

19. The medicament of claim 18, wherein the amount of the active ingredient is in the range of from about 1.0 to 180 mg/kg/day.

20. The medicament claim 18, wherein the medicament is for intravenous injection or given orally.

21. The medicament of claim 20, wherein the medicament exerts its action, at least in part via activation of soluble guanylyl cyclase.

22. The medicament of claim 17, wherein the active ingredient is selected from 1,7-dioxaspiro[5.5]undecane and 1,7-dioxaspiro[5.5]undec-2-ene, 1,7-dioxaspiro[5.5]undec-3-ene, 1,7-dioxaspiro[5.5]undec-4-ene, 1,7-dioxaspiro[5.5]undecan-4-ol and 1,7-dioxaspiro[5.5]undecan-5-ol.
